(19) United States
(12) Patent Application Publication
(10) Pub. No.: US 2016/0100843 A1
(43) Pub. Date: Apr. 14, 2016

(54) MEDICAL DEVICE FOR CLOSING AN OPENING OR ISOLATING A STRUCTURE IN THE CARDIOVASCULAR SYSTEM

(71) Applicant: Basilios Eleftherios Sideris, San Francisco, CA (US)

(72) Inventor: Basilios Eleftherios Sideris, San Francisco, CA (US)

(21) Appl. No.: 14/740,251

(22) Filed: Jun. 15, 2015

Related U.S. Application Data
(60) Provisional application No. 62/012,424, filed on Jun. 16, 2014.

(51) Int. Cl. A61B 17/12; A61M 25/01; A61M 25/10

(52) U.S. Cl. CPC A61B 17/12136; A61B 17/1204; A61B 17/12109; A61M 25/10186; A61M 25/0169; A61B 2017/12127

(57) ABSTRACT

An apparatus for closure of an opening or isolation of a structure in the cardiovascular system. The apparatus generally includes an occluding segment, a delivery segment and a control segment. The occluding segment may be composed of one or more independently inflatable members which are detachably or permanently attached to a distal portion of the delivery segment. The occluding segment may contain a stem-like member, which constrains expansion of the inflatable member in one or more directions. The apparatus is intended to enable a combination of the following: inflation of the occluding segment, ‘over the wire’ advancement, detachment of a portion of the device, and steerability. Finally, the control segment may contain components such as ports, handles, and other mechanisms which enable safe and effective implantation of the device.

### RELATED APPLICATION DATA

This application claims the priority of prior provisional U.S. patent application Ser. No. 62/012,424 filed on Jun. 16, 2014, which application is hereby incorporated by reference herein in its entirety.

### FIELD OF THE INVENTION

This invention relates generally to cardiovascular devices intended for closure of an opening or isolation of a structure in the cardiovascular system. More specifically, this document discloses significant improvements in minimally invasive devices and methods for closure of cardiovascular structures that adversely affect the proper flow of blood.

### BACKGROUND OF THE INVENTION

In the presence of certain cardiovascular structures, blood flow can become modified from its normal flow patterns, potentially resulting in serious risks to patient health. Such structures may include congenital or acquired abnormalities that affect a relatively small subset of the population. Alternatively, such structures can include those that are commonly present and only become pathogenic under certain conditions. Examples of the former are congenital heart defects including Atrial Septal Defect (ASD), Ventricular Septal Defect (VSD), and Patent Ductus Arteriosus (PDA), and examples of the latter are structures such as Patent Foramen Ovale (PFO) and Left Atrial Appendage (LAA). Congenital heart defects can cause an abnormal distribution of cardiovascular pressures and the dilution of oxygenated blood, which can significantly reduce cardiac pumping efficiency. Normal cardiac structures can sometimes become pathogenic by functioning as a location of blood clot formation, as in the LAA, or as a path by which blood clots can travel to the brain, as in PFO. Furthermore, certain medical procedures can create residual structures, either unintentionally or deliberately, which are undesirable. These can include residual tunnels surrounding prosthetic valves, as well as the openings left by access sheaths after a cardiovascular procedure. All of the above mentioned structures are generally characterized by a hole or opening, resulting in leaks or fluid exchange between cardiac chambers and/or blood vessels.

One treatment option for such cardiovascular structures is to occlude the communication using a surgically or percutaneously implanted device. Devices used for this purpose generally include frames which are relatively rigid compared to cardiovascular tissue. These frames are often composed of metallic materials that are intended to provide support indefinitely and maintain the device in place. Despite being successful in many ways, concerns related to persisting rigid frames remain. These may include wire fractures or interference with sensitive cardiovascular structures such as valves or nodes of the cardiac conduction system. Additionally, in cases where the relative motion of the device is different to that of adjacent cardiac structures, erosions can occur. An improved device could include a less rigid supporting structure such as that of a compliant balloon. A balloon based closure device is self-centering relative to the structure intended for closure and there is limited overhang of the occluder onto adjacent tissues, reducing the risk of interference with sensitive neighboring structures. Another improvement could include a supporting structure which partly or completely degrades over time or is otherwise diminished, at which point the original forces providing support would decrease. Additionally, a feature which promotes tissue ingrowth may be included. As the supporting structure degrades, ingrown tissue would take over the function of maintaining any remaining device materials in place.

Another drawback with commonly used closure devices is the use of hooks or barbs to accomplish secure attachment to cardiac tissue. Such features can potentially be traumatic, especially in cases where the targeted cardiac structure is anatomically diverse among patients or in cases where there are unexpected events such as embolizations. A device which adequately conforms to the shape of the targeted cardiac structure and relies on distention or stretching for secure attachment could provide an important improvement over currently used devices.

Finally, closure devices are normally deployed through delivery catheters, the ends of which are placed adjacent to the cardiovascular structure intended for closure. In certain instances the operator may not be able to position the delivery catheter appropriately, making device delivery difficult or impossible. Furthermore the delivery catheter may be positioned in a cardiovascular chamber where pressures are negative relative to atmospheric pressures. This increases the risk of air being drawn into the cardiovascular system, causing gas embolism. An improved device could avoid the use of a long delivery sheath and be delivered independently using a guidewire as a rail in an ‘over the wire’ procedure.

### SUMMARY OF THE INVENTION

This document discloses novel technical enablements of the improvements discussed in the ‘background of the invention’ section. Devices envisioned by this invention generally contain the following segments: occluding segment, delivery segment, control segment. The occluding segment may be composed of one or more independently inflatable members which are detachably or permanently attached to a distal portion of the delivery segment. The inflatable members may be surrounded by a patch made of an elastic, biocompatible material. The occluding segment may contain a stem-like member, which constrains expansion of the inflatable member in one or more directions. Optionally, the occluding segment could be formed in a way that is able to accommodate a wire through one of its axes. The delivery segment may be comprised of one or more components that enable some or all of the following: inflation of the occluding segment, ‘over the wire’ advancement, detachment of a portion of the device, and steerability. Finally, the control segment may contain components such as ports, handles, and other mechanisms which enable safe and effective implantation of the ii device. All materials used in the devices envisioned by this invention must be biocompatible and may be biodegradable/bioabsorbable under normal physiological conditions.

### BRIEF DESCRIPTION OF THE DRAWINGS

The present invention is best understood with reference to the ‘detailed description of preferred embodiments of the invention’ when read in conjunction with the attached drawings, in which like numerals refer to like elements, and in which:

FIG. 1 is a side view of a delivery sheath and a device which includes an occluding segment, delivery segment, and control segment. The occluding segment contains a detachable single balloon member surrounded by a patch;

FIG. 2 is a side view of a delivery sheath and a device which includes an occluding segment, delivery segment, and control segment. The occluding segment contains a detachable double balloon member surrounded by a patch;

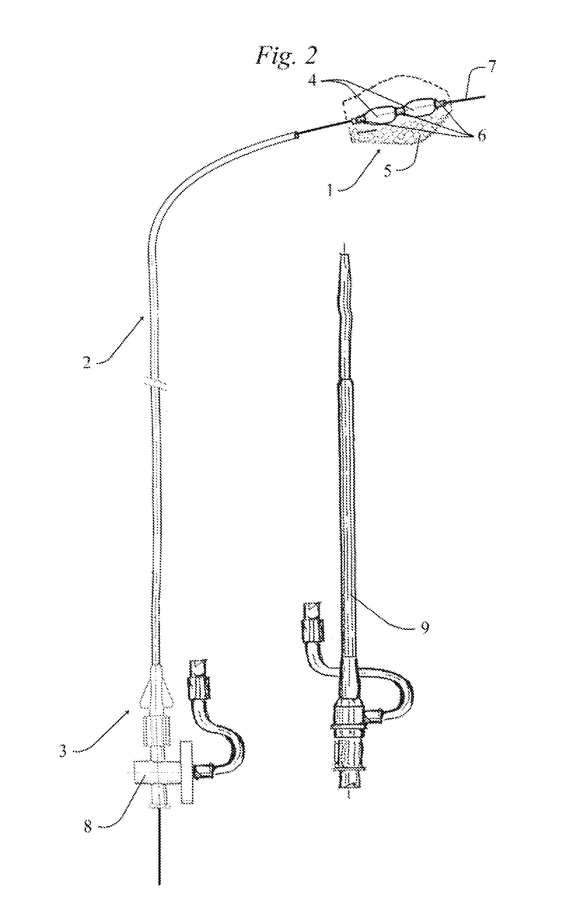

FIG. 3 is a side view of a delivery sheath and a device which includes an occluding segment, delivery segment, and control segment. The occluding segment contains a single balloon member which is rigidly attached to the distal tip of the delivery segment and is surrounded by a patch;

### DETAILED DESCRIPTION OF PREFERRED EMBODIMENTS OF THE INVENTION

In the disclosure that follows, in the interest of clarity, not all features of actual implementations are described. It will of course be appreciated that in the development of any such actual implementation, as in any such project, numerous engineering and technical decisions must be made to achieve the developers' specific goals and subgoals (e.g., compliance with system and technical constraints), which will vary from one implementation to another. Moreover, attention will necessarily be paid to proper medical and engineering practices for the environment in question. It will be appreciated that such development efforts might be complex and time-consuming, outside the knowledge base of typical laymen, but would nevertheless be a routine undertaking for those of ordinary skill in the relevant fields.

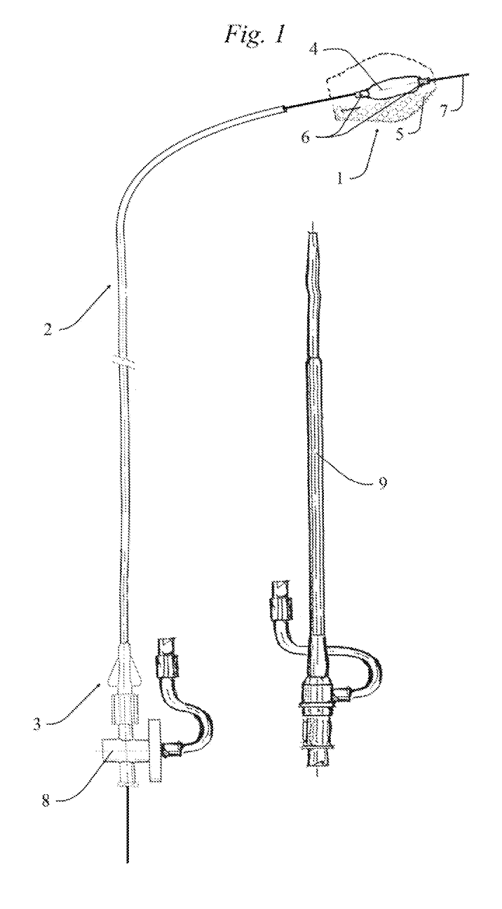

The present invention encompasses devices and methods of implantation thereof for closure of cardiovascular structures that are adversely affecting the proper flow of blood. In accordance with one embodiment of the invention, the device includes an occluding segment 1, a delivery segment 2, and a control segment 3 as seen in FIGS. 1 & 2. The occluding segment is comprised of one or more balloon members 4 which can be detachably coupled to the delivery segment. A patch 5 may be attached to one or more points on the balloon member(s). The occluding segment may include one or more valves in order to maintain a pressure differential between balloon members as well as between balloon members and the external environment. In one configuration, each balloon member could be independently inflated/deflated through a dedicated lumen and a valve could be positioned at the proximal end of each balloon member. In another configuration, multiple balloon members could be independently inflated/deflated through the same lumen. A valve could be positioned at the proximal end of the most proximal balloon member and valve/gasket components 6 could be positioned between adjacent balloon members. Inflation/deflation of each balloon member could be achieved by crossing one or more valve/gasket components (including the proximal valve) with a dedicated inflation catheter, through which fluid could either be injected or removed. The dedicated inflation catheter can optionally be capped at its end and have at least one set of holes along the length of its tip. When attempting to inflate/deflate two adjacent balloon members, the tip of the inflation catheter could be placed through the joining valve/gasket component and positioned in a way that exposes the holes to one balloon member at a time. The position of the inflation catheter could then be changed to inflate the other balloon member. In another configuration, the end of an inflation catheter could be temporarily attached to the base of the proximal valve, allowing fluid to pass from the catheter and through the valve. In this case, fluid could not be removed from the balloon member without using a second smaller catheter to cross the valve. In another configuration, balloon members could be attached to one another without valve/gasket components, in which case they could be inflated simultaneously through the same inflation catheter. In all of these configurations, another one-way valve could be positioned at the distal end of the most distal balloon member. An ‘over the wire’ procedure could be performed by passing a wire 7 through the distal valve and advancing it through the proximal side of the occluding segment either directly through the intermittent valve/gasket components and finally the proximal valve, or through the tip of an inflation catheter which has been positioned inside the occluding member. The balloon members may be inflated with the wire in place, enabling deflation and simple device repositioning if necessary. Alternatively, balloon members could be rigidly attached at two or more points along a stem-like member which could include one or more lumens traversing at least a portion of the stem-like member (i.e. a lumen that is capped on one end). These lumens may include one or more holes providing access to one or more balloon members. Valves could be attached directly to the stem-like member in order to achieve inflation or deflation of the balloon members, and advancement of a wire through the balloon members. The stem-like member may also include one or more portions with different elastic modulus along its length. This would allow, for example, a flexible neck that would accommodate relative movement between two adjacent balloon portions. Inflation, deflation, and advancement of a wire could be achieved through the same lumen, similarly to the configurations which do not include a stem-like member. Alternatively, separate lumens could be used for advancement of a wire and inflation/deflation of the balloon members. Finally, the occluding segment could include a feature which allows it to be detachably coupled to the delivery segment. This feature may include one or more of the following: lumen, threaded lumen, barb, gap, groove, hole. These parts would be used in conjunction with a mechanism on the distal part of the delivery segment that enables the occluding segment to be altered between two configurations, attached and detached. The mechanism could include a wire with a threaded tip which passes through the delivery segment and screws into a threaded lumen on the stem-like member. Alternatively the mechanism could include a non-threaded wire which passes through the delivery segment and into a non-threaded lumen on the stem-like member. Additionally, a thread would be looped around the wire through a hole on the side of the stem-like member. Pulling the wire would release the thread and in that way detach the occluding segment. Alternatively, a thread could be passed through the delivery segment and tightened around a bulb, gap, or groove on the occluding segment. In order to achieve detachment, the thread would be loosened and removed. The mechanism could also serve to maintain proper alignment between the occluding and delivery segments. The delivery segment could include one or more independent or connected lumens which may be aligned with the lumens in the occluding segment. At least one lumen would be used for inflation or deflation of the balloon member. Such a lumen could optionally accommodate a catheter which could be independently advanced to or retracted from the occluding member. In the advanced position, where the tip of the catheter has crossed one or more valve/gasket components in the occluding segment, both inflation and deflation could be achieved by injecting or removing fluid through the catheter. Such a lumen could also accommodate a wire. Alternatively, a dedicated lumen could be used for wire advancement. The curvature of the delivery segment could be set in a way that enables smooth advancement to the target cardiovascular structure. For example, an L shaped distal end could facilitate advancement to the Left Atrial Appendage and a J shaped distal end could facilitate advancement to the Ventricular Septum. A steerable component could be added to the delivery segment, either within one or more lumens as a steerable wire or surrounding one or more lumens as a steerable catheter. The delivery segment would be attached to the control segment which would remain outside of the patient during device implantation. The control segment could include ergonomic controls such as push, pull, or rotary mechanisms to deliver, detach, or retrieve the occluding member. Additionally it could include one or more ports 8 for inflation/deflation of the occluding member(s), as well as controls to steer the device into place. Any device components which provide for a conduit between the cardiovascular system and the external environment may incorporate valves or gaskets in order to prevent air embolism.

Implantation of the first embodiment of this invention may be carried out according to the following steps. Access to the cardiovascular system may be gained using commonly used methods. A wire may be advanced to the cardiovascular structure ii targeted for repair. A short or long sheath 9 able to accommodate the device described in this invention may be advanced over the wire into the cardiovascular system. The portion of the wire which extends outside of the patient may then be passed through the distal tip of the device and advanced through to its proximal end. The device may then be advanced over the wire, through the sheath, and to the cardiovascular structure targeted for repair. If available, steerability may be used during device advancement as necessary. The device would then be inflated, detached, and the wire withdrawn. Withdrawal of the wire could also be achieved prior to detachment or prior to inflation. When retracting components through the occluding segment after it has been detached from the delivery segment, an additional counter force could be provided by positioning the tip of the delivery segment on the proximal end of the occluding segment. The delivery sheath would then be retracted and the percutaneous entry point would be sutured.

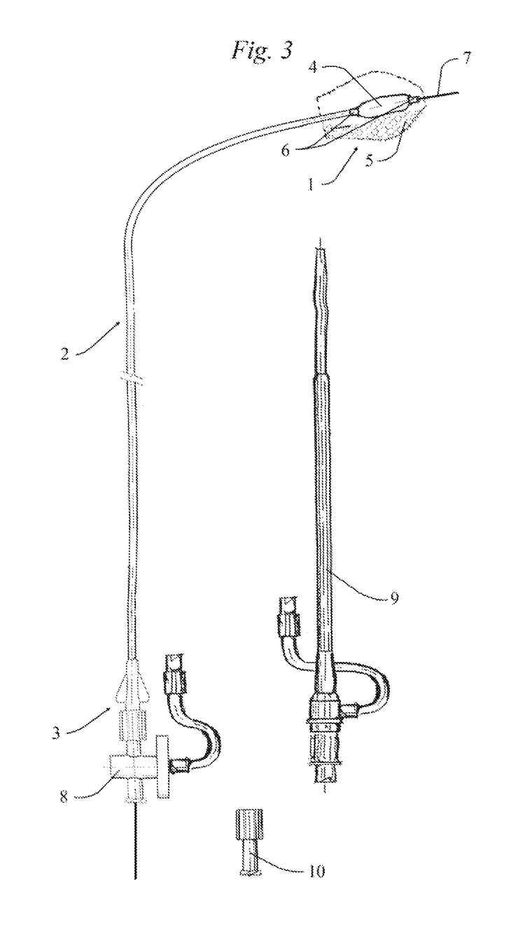

In accordance with a second embodiment of the invention, the device includes an occluding segment, a delivery segment, and a control segment as seen in FIG. 3. The occluding segment is rigidly attached to at least a portion of the delivery segment and is comprised of one or more balloon members. A patch may be attached to one or more points on the balloon member(s). The occluding segment may include one or more valves in order to maintain a pressure differential between balloon members as well as between balloon members and the external environment. In one configuration, each balloon member could be independently inflated/deflated through a dedicated lumen. A valve could be positioned at the proximal end of each balloon member. In another configuration, multiple balloon members could be independently inflated/deflated through the same lumen. A valve could be positioned at the proximal end of the most proximal balloon member and valve/gasket components could be positioned between adjacent balloon members. Inflation/deflation of each balloon member could be achieved by crossing one or more valve/gasket components (including the proximal valve) with a dedicated inflation catheter, through which fluid could either be injected or removed. The dedicated inflation catheter could optionally be capped at its end and have at least one set of holes along the length of its tip. When attempting to inflate/deflate two adjacent balloon members, the tip of the inflation catheter could be placed through the joining valve/gasket component and positioned in a way that exposes the holes to one balloon member at a time. The position of the inflation catheter could then be changed to inflate the other balloon member. In another configuration, the end of an inflation catheter could be rigidly attached to the base of the proximal valve, allowing fluid to pass from the catheter and through the valve. In this case, fluid could not be removed from the balloon without using a second smaller catheter to cross the valve. In another configuration, balloon members could be attached to one another without valve/gasket components, in which case they could be inflated simultaneously through the same inflation catheter. In all of these configurations, another valve could be positioned at the distal end of the most distal balloon member. An ‘over the wire’ procedure could be performed by passing a wire through the distal valve and advancing it through the proximal side of the occluding segment either directly through the intermittent valve/gasket components and finally the proximal valve, or through the tip of an inflation catheter which has been positioned inside the occluding member. The balloon members may be inflated with the wire in place, enabling deflation and simple device repositioning if necessary. Alternatively, balloon members could be rigidly attached at two or more points along a stem-like member which could include one or more lumens traversing at least a portion of the stem-like member (i.e. a lumen capped on one end). The stem-like member could extend proximally beyond the balloon member(s) and form part or all of the delivery segment of the device. Lumens contained in the stem-like member may include one or more holes providing access to one or more balloon members. Valves could be attached directly to the stem-like member in order to achieve inflation or deflation of the balloon members, and advancement of a wire through the balloon members. Inflation, deflation, and advancement of a wire could be achieved through the same lumen, similarly to the configurations which do not include a stem-like member. Alternatively, separate lumens could be used for advancement of a wire and inflation/deflation of the balloon members. The delivery segment could include one or more independent or connected lumens which may be aligned with the lumens in the occluding segment. At least one lumen would be used for inflation or deflation of the balloon member. Such a lumen could optionally accommodate a catheter which could be independently advanced to or retracted from the occluding member. In the advanced position, where the tip of the catheter has crossed one or more valve/gasket components in the occluding segment, both inflation and deflation could be achieved by injecting or removing fluid through the catheter. Such a lumen could also accommodate a wire. Alternatively, a dedicated lumen could be used for wire advancement. The curvature of the delivery segment could be set in a way that enables smooth advancement to the target cardiovascular structure. For example, an L shaped distal end could facilitate advancement to the Left Atrial Appendage and a J shaped distal end could facilitate advancement to the Ventricular Septum. A steerable component could be added to the delivery segment, either within one or more lumens as a steerable wire or surrounding one or more lumens as a steerable catheter. The delivery segment would be attached to the control segment which would remain outside of the patient during device implantation. The control segment could include ergonomic controls such as push, pull, or rotary mechanisms to deliver, detach, or retrieve the occluding member. Additionally it could include one or more ports for inflation/deflation of the occluding member, as well as controls to steer the device into place. Detachment of the device could be achieved by clamping the delivery segment at the percutaneous entry point and disconnecting it from the control segment. An anchoring component 10 which may include a clamping mechanism as well as a distal sponge-like material to facilitate hemostasis could then be applied on the delivery segment, directly at the percutaneous entry point. Any device components which provide for a conduit between the cardiovascular system and the external environment may incorporate valves or gaskets in order to prevent air embolism.

Implantation of the second embodiment of this invention may be carried out according to the following steps. Access to the cardiovascular system may be gained using commonly used methods. A wire may be advanced to the cardiovascular structure targeted for repair. A short or long sheath able to accommodate the device described in this invention may be advanced over the wire into the cardiovascular system. The portion of the wire which extends outside of the patient may then be passed through the distal tip of the device and advanced through to its proximal end. The device may then be advanced over the wire, through the sheath, and to the cardiovascular structure targeted for repair. If available, steerability may be used during device advancement as necessary. The device would then be inflated, and the wire withdrawn. Withdrawal of the wire could also be achieved prior to inflation. The delivery sheath would then be retracted beyond the percutaneous entry point and the extending delivery segment of the device would be clamped. The anchoring mechanism would then be applied to delivery segment, directly on the percutaneous entry point. The delivery segment extending beyond the anchoring mechanism would then be cut and the skin would be sutured over the anchor.

What is claimed is:

1. An occlusive device for placement in the cardiovascular system, comprising:
one or more inflatable member(s), each having a proximal and distal end, and containing at least one valve;
a catheter portion with two or more lumens, each having a proximal and distal end; and
at least one elongate member, movably disposed within each lumen.

2. An occlusive device in accordance with claim 1, further comprising a patch, made of an elastic material, covering said inflatable member(s).

3. An occlusive device in accordance with claim 1, wherein at least one stem-like member is attached to the distal and proximal ends of each inflatable member.

4. An occlusive device in accordance with claim 3, wherein said stem-like member contains one or more lumens.

5. An occlusive device in accordance with claim 3, wherein said stem-like member contains one or more valves and one or more transversely placed holes.

6. An occlusive device in accordance with claim 3, wherein said stem-like member contains portions of different elastic modulus.

7. An occlusive device in accordance with claim 3, whereby following deployment of the device, an anchoring component is attached to a portion of said stem-like member which extends beyond said inflatable member(s).

8. An occlusive device in accordance with claim 1, wherein said elongate member is used to removably attach said catheter portion to one or more said inflatable member(s).

9. An occlusive device in accordance with claim 1, wherein said elongate member is a thread, wire, or catheter.

10. An occlusive device in accordance with claim 9, wherein said elongate member is steerable.
